(19) United States
(12) Patent Application Publication
(10) Pub. No.: US 2017/0281828 A1
(43) Pub. Date: Oct. 5, 2017

(54) THREE-DIMENSIONAL BIOPRINTED ARTIFICIAL CORNEA

(71) Applicant: THE REGENTS OF THE UNIVERSITY OF CALIFORNIA, Oakland, CA (US)

(72) Inventors: Kang ZHANG, La Jolla, CA (US); Shaochen CHEN, San Diego, CA (US); Xin QU, Medford, MD (US); Hong OUYANG, San Diego, CA (US)

(21) Appl. No.: 15/513,938

(22) PCT Filed: Sep. 24, 2015
(86) PCT No.: PCT/US15/51999

§ 371 Date: Mar. 23, 2017

Related U.S. Application Data
(60) Provisional application No. 62/054,924, filed on Sep. 24, 2014.

(51) Int. Cl. A61L 27/38; B33Y 80/00; C12N 5/079; B33Y 10/00; A61L 27/52; A61L 27/48

(52) U.S. Cl. CPC A61L 27/3891; A61L 27/3808; A61L 27/3813; A61L 27/3834; A61L 27/52; A61L 27/48; C12N 5/0621; B33Y 10/00; B33Y 80/00; A61L 2430/16; A61L 2400/12; C12N 2533/54; C12N 2533/30; C12N 2513/00

(57) ABSTRACT

An artificial cornea is fabricated by separately culturing live stromal cells, live corneal endothelial cells (CECs) and live corneal epithelial cells (CEpCs), and 3D bioprinting separate stromal, CEC and CEpC layers to encapsulate the cells into separate hydrogel nanomeshes. The CEC layer is attached to a first side of the stromal layer and the CEpC layer to a second side of the stromal layer to define the artificial cornea.

### RELATED APPLICATIONS

This application claims the benefit of the priority of U.S. Provisional Application No. 62/054,924, filed Sep. 24, 2014, which is incorporated herein by reference in its entirety.

### FIELD OF THE INVENTION

The invention relates to 3D bioprinting of artificial tissue and more specifically to an artificial cornea produced using 3D bioprinting.

### BACKGROUND OF THE INVENTION

Disease or damage to one or more layers of the cornea can lead to blindness that is commonly treated by corneal transplant. Approximately 40,000 patients undergo corneal transplant surgery in the United States every year. The vast majority of these people receive a replacement cornea from a human donor. Although the surgery has a high success rate, the supply of donor tissue is limited, and wait lists can be long. In the developing world, access to donor tissue is even more difficult. Further, while human donor transplants are the standard treatment for corneal blindness, the complications and limitations inherent in them have prompted development of synthetic corneal substitutes. Existing synthetic corneas can be categorized into: 1) fully synthetic prostheses (e.g., keratoprostheses) and 2) hydrogels that permit regeneration of the host tissue.

Keratoprostheses, or Kpros, the best-known artificial corneas, perform the refractive function of the cornea. Although Kpros have been available for many years in various forms, the fabrication of synthetic stromal equivalents with the transparency, biomechanics, and regenerative capacity of human donor corneas remain a formidable challenge. Further, the application of keratoprostheses is impeded by the complicated implantation procedures and major post-surgical complications, including infection, calcification, retroprosthetic membrane formation and glaucoma. In some cases, due to their propensity for infection, patients must take a lifelong course of antibiotics. As a result, the artificial cornea is used only as a last resort in patients who have repeatedly rejected natural donor tissue or who are otherwise not eligible for such transplant surgery.

The second type of engineered corneas are synthetic hydrogel-based, cell-free implants, which are designed to recruit host cells to grow an epithelial layer on the implant's surface and restore functionality. Many of these hydrogel implants resemble organic tissue and have a high elastic modulus with desirable optical properties. However, in most cases, mechanical or biological fixation is problematic—integration of the implanted scaffold with the host tissue is an extremely time-consuming process. This slow time-course is further exacerbated by the limited cell repopulation activity in patients who are older and/or severely injured. In addition, some of these hydrogel implants have reportedly become partially biodegraded after long-term implantation, leading to loss of transparency and failure of the grafting. Attempts to address some of the problems with cell-free implants include incorporation of glucosaminoglycans in the hydrogel matrix, which are believed to be necessary for cell adhesion and modulation of degradability.

One of the transformative applications of bionanotechnology is to create revolutionary approaches for the reconstruction and regeneration of human tissues and organs. This promise is based on the powerful capability that nanotechnology provides in a biological context: unique modalities of control over cellular machinery at the nanoscale. Due to their special surface characteristics, subcellular length scales, and precisely directed modular architectures, nanostructures and their incorporation within tissue engineering constructs serve new paradigms for regenerative medicine. 3D bioprinting—which uses biomaterials, cells, proteins, and other biological compounds as building blocks to fabricate 3D structures through additive manufacturing processes—offers novel approaches that can accelerate the realization of anatomically correct tissue constructs for transplantation. This collection of emerging technologies and their synergistic integration—by providing nanotechnology-enabled 3D tissue models that mimic normal and pathological physiology—can not only redefine the clinical capabilities of regenerative medicine but also transform the toolsets available for drug discovery and fundamental research in the biological sciences.

An approach to overcome drawbacks that are being experienced with existing artificial cornea technologies would be to provide a tissue-engineered cell-based corneal substitute that resists rejection and is easily integrated with host tissue. The present invention is directed to such an approach.

### BRIEF SUMMARY

In an exemplary embodiment, a method and system are provided for fabrication of cell-laden corneal substitutes using a 3D bioprinting platform. Such artificial corneas provide a new approach that avoids many of the complications involved in existing methods for treatment of corneal epithelial disease. According to an embodiment of the invention, 3D bioprinters allow for cell encapsulation within a printed network, enabling live printing of tissue structures with micro- and nanometer scale resolution. The cell-laden corneal substitutes can shorten the time for transplants to integrate with host tissue. Further, the digital (i.e., customizable) nature of 3D printing allows one to develop patient-specific tissue models with designed shape and curvature. Such 3D-printed cornea tissues will have immediate applications in clinical transplantation, human ocular surface disease modeling (e.g., for dry eye diseases), early drug screening to replace or reduce the need for animal testing, and in drug efficacy testing for wound healing.

According to an exemplary embodiment, an artificial cornea is fabricated by separately culturing live stromal cells, live corneal endothelial cells (CECs) and live corneal epithelial cells (CEpCs), and 3D bioprinting separate stromal, CEC and CEpC layers to encapsulate the live cells into separate hydrogel nanomeshes. The CEC layer is attached to a first side of the stromal layer and the CEpC layer to a second side of the stromal layer to define the artificial cornea.

In one aspect of the invention, a method for fabricating an artificial cornea, comprises culturing live stromal cells; 3D bioprinting a stromal layer encapsulating the live stromal cells into a first hydrogel nanomesh; culturing live corneal endothelial cells (CECs); 3D bioprinting a CEC layer encapsulating the live CECs into a second hydrogel nanomesh; culturing live corneal epithelial cells (CEpCs); 3D bioprinting a CEpC layer encapsulating the live CEpCs into a third hydrogel nanomesh; and attaching the CEC layer to a first side of the stromal layer and the CEpC layer to a second side of the stromal layer. In some embodiments the steps of culturing are performed in parallel. The steps of 3D bioprinting the CEC layer and the CEpC layers may be performed in parallel. The CEC layer may be attached to the first side of the stromal layer by sequentially printing the stromal layer and the CEC layer. Alternatively, the CEC layer may be attached to the first side of the stromal layer by applying a thin film of hydrogel between each of the layers and curing via UV exposure. The CEpC layer may be attached to the second side of the stromal layer by applying a thin film of hydrogel between each of the layers and curing via UV exposure. In a preferred embodiment, prior to 3D bioprinting the CEC layer, the CECs are mixed with a prepolymer solution of acryloyl-polyethylene glycol (PEG)-collagen. The prepolymer solution may further include methacrylated hyaluronic acid (MA-HA). In another preferred embodiment, prior to 3D bioprinting the CEpC layer, the CEpCs are mixed with a prepolymer solution of acryloyl-PEG-collagen. The prepolymer solution may further include MA-HA. In another preferred embodiment, prior to 3D bioprinting the stromal layer, encapsulating the stromal cells in an acryloyl-PEG-collagen hydrogel, which may further include MA-HA. The stromal cells may be encapsulated at a cell density in the range of around 5 million/ml to 25 million/ml stromal cells.

In some embodiments, the live CEpCs are cultured and differentiated from limbal stem cells (LSCs). The LSCs may be obtained from autologous tissue. The live CECs may be cultured and differentiated from CEC progenitors from a human donor. The CEC progenitors may be obtained from autologous tissue.

In another aspect of the invention, an artificial cornea comprises a layered structure comprising a 3D bioprinted stromal layer comprising live stromal cells encapsulated into a first hydrogel nanomesh, the stromal layer having a first side and a second side; a 3D bioprinted CEC layer comprising live CECs encapsulated into a second hydrogel nanomesh; and a 3D bioprinted CEpC layer comprising live CEpCs encapsulated into a third hydrogel nanomesh; wherein the CEC layer is attached to the first side of the stromal layer and the CEpC layer is attached to the second side of the stromal layer. In some embodiments of the artificial cornea, one or more of the CEC layer and the CEpC layer is attached by a thin film of hydrogel applied between the layers and cured via UV exposure.

The live stromal cells are preferably encapsulated into a hydrogel prior to bioprinting the stromal layer. The hydrogel may be acryloyl-PEG-collagen, and may further include MA-HA. The live CECs are also encapsulated into a hydrogel prior to bioprinting the CEC layer. The hydrogel may be acryloyl-PEG-collagen, and may further include MA-HA. The live CEpCs are also encapsulated into a hydrogel prior to bioprinting the CEpC layer. The hydrogel may be acryloyl-PEG-collagen, and may further include MA-HA. The live CEpCs may be obtained from cultured and differentiated LSCs.

By integrating the emerging technologies in the multidisciplinary domains of nanotechnology, 3D bioprinting, and regenerative medicine, we have developed artificial corneas to change the clinical landscape by eliminating the current dependency on corneal donor tissue and by providing a new strategy for restoring vision that would otherwise be lost in human patients with severe corneal blindness. The native, multilaminar anatomy of the cornea is well suited as an initial application of our layer-by-layer nanomesh integrated 3D printing approach.

### BRIEF DESCRIPTION OF THE DRAWINGS

FIG. 1 is a schematic diagram of an embodiment of the 3dLP printing system.

FIG. 2 is a schematic diagram of an embodiment of an artificial cornea created using 3D live printing in comparison with a human analog.

FIG. 3 is a flow chart of an exemplary process for fabricating an artificial cornea according an embodiment of the invention.

FIG. 4A shows rabbit corneas after cell transplantation with LSCs cultured on gelatin methacrylate (GelMA) based matrix showing typical corneal epithelium histology and smooth and transparent cornea surface without epithelial defects, where the left panel shows H&E stain and the right panel is a white light micrograph of the cornea.

FIG. 4B shows the denuded cornea covered with a human amniotic membrane only, showing histology of epithelial metaplasia and opaque cornea with vascularization.

FIG. 4C shows a rabbit cornea 3 months post transplantation.

FIGS. 5A-C show various microstructures created by 3D bioprinting, where FIG. 5A shows a multi-layer log-pile scaffold with 200 μm pore size using PEGDA; FIG. 6B shows a 3D-printed vasculature-like microstructure in GelMA (scale bar=30 μm); and FIG. 6C shows 10T1/2 cells encapsulated in a GelMA scaffold (scale bar=1 mm).

FIG. 6 illustrates an exemplary synthesis scheme of GelMA hydrogels.

FIG. 7 shows a confluent CEC layer created using the 3dLP system.

FIGS. 8A-C illustrate an assessment of optical property of the hydrogel films with different compositions.

FIGS. 9A-9C show the gradual recovery of clarity and functionality of a transplanted cornea, at day 5, day 10 and day 15 post transplantation, respectively.

FIG. 10 is a flow chart of an exemplary process for designing, fabricating and transplanting an artificial cornea according to an embodiment of the invention.

### DETAILED DESCRIPTION

By integrating the emerging technologies in the multidisciplinary domains of nanotechnology, 3D bioprinting, and regenerative medicine, we have developed artificial corneas to change the clinical landscape by eliminating the current dependency on corneal donor tissue and by providing a new strategy for restoring vision that would otherwise be lost in human patients with severe corneal blindness. The inventive approach utilizes nano-based 3D printing for corneal regeneration. The native, multilaminar anatomy of the cornea is well suited as an initial application of our layer-by-layer nanomesh integrated 3D printing approach.

The 3D live printing (“3dLP”) technology utilizes continuous 3D printing of a series of layers by way of digital micromirror device (DMD) projection and an automated stage. Similar 3D printing systems have been previously disclosed for different applications. (See, e.g., International Publication No. WO2014/197622, and International Publication No. WO2012/071477, which are incorporated herein by reference).

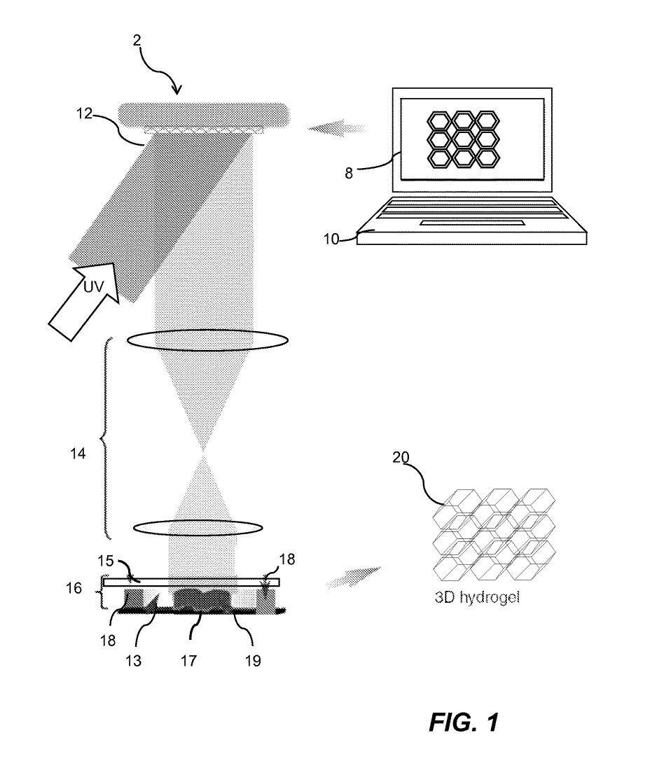

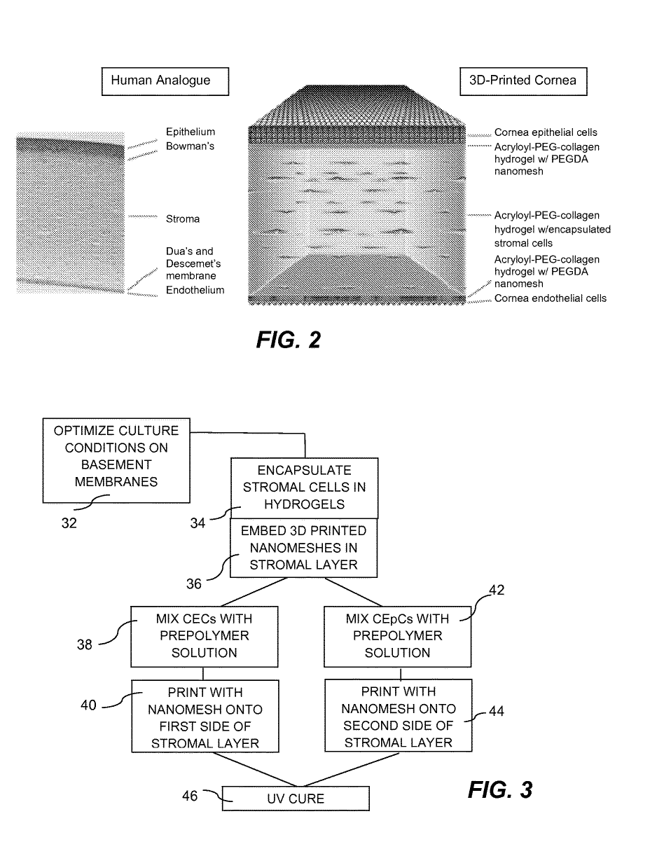

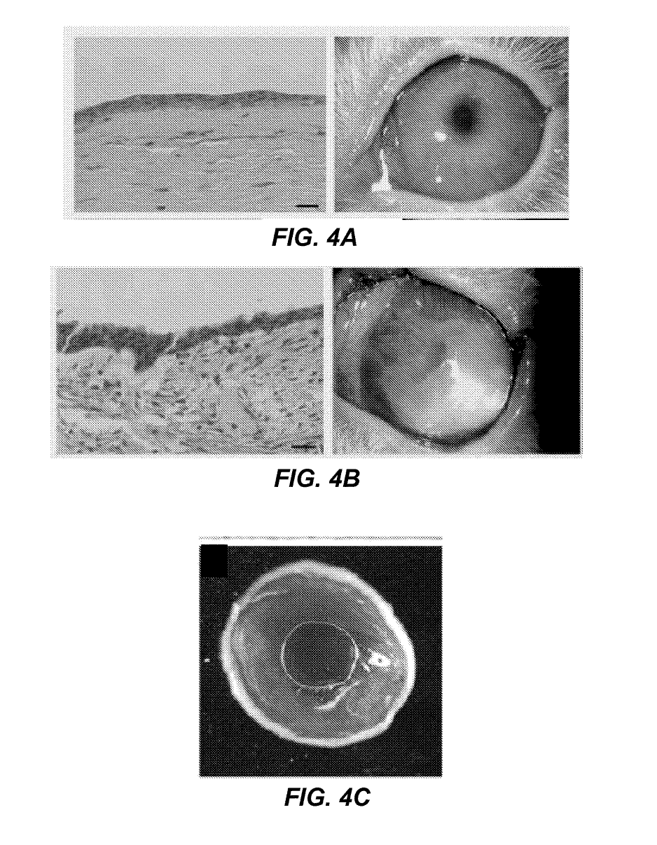

Fabrication of an artificial cornea using a 3D hydrogel matrix employs digital mask (i.e., “maskless”) projection printing in which a digital micro-mirror device (DMD) found in conventional computer projectors to polymerize and solidify a photosensitive liquid prepolymer using ultraviolet (UV) or other light sources appropriate for the selected polymer. FIG. 1 illustrates an exemplary implementation of a maskless projection printing system 2, referred to as the “dynamic projection stereolithography” (DPsL) platform. The “maskless” or digital mask approach allows for the use of controllable and interchangeable reflected light patterns rather than static, more expensive physical masks like those used in conventional photolithography. The system 2 includes a UV light source 6, a computer controller 10 for sliced image flow generation to guide creation of the pattern, a DMD chip 12, which is composed of approximately one million micro-mirrors, embedded in a projector as a dynamic mask, projection optics 14, a translation stage 16 for sample position control, and a source of photocurable prepolymer material 13. The DMD chip 12 acts an array of reflective coated aluminum micro-mirrors mounted on tiny hinges that enable them to tilt either toward the light source or away from it, creating a light (“on”) or dark (“off”) pixel on the projection surface., thus allowing it to redirect light in two states [0,1], tilted with two bias electrodes to form angles of either +12° or −12° with respect to the surface. In this way, a DMD system can reflect pixels in up to 1,024 shades of gray to generate a highly detailed grayscale image.

The computer controller 10 may display an image of the desired structure 8 for a given layer, as shown, and/or may display the desired parameters of the matrix. A quartz window or other light transmissive material 15, spacers 18, and base 19, all supported on the translation stage 16, define a printing volume or “vat” containing the prepolymer solution 13. Additional solution 13 may be introduced into the printing volume as needed using a syringe pump (not shown.) Based on commands generated by controller 10, the system spatially modulates collimated UV light using DMD chip 12 (1920×1080 resolution) to project custom-defined optical patterns onto the photocurable prepolymer solution 13.

To generate 3D structures, projection stereolithography platforms such as DPsL employ a layer-by-layer fabrication procedure. In an exemplary approach, a 3D computer rendering (made with CAD software or CT scans) is deconstructed into a series of evenly spaced planes, or layers. For purposes of illustration, a simple honeycomb pattern representing one layer of a desired mesh-like structure is displayed on display 8 of computer controller 10. The pattern for each layer is input to the DMD chip 12, exposing UV light onto the photocurable (pre-polymer) material 13 to create a polymer structure 17. After one layer is patterned, the computer controller 10 lowers the automated stage 16 and the next pattern is displayed to build the height of the polymer structure 17. Through programming of the computer controller 10, the user can control the stage speed, light intensity, and height of the structure 17, allowing for the fabrication of a variety of complex structures 20. It should be noted that while a single honeycomb structure is illustrated, any combination of patterns, may be used to construct multi-layer structures of different patterns overlying each other.

As an alternative to the DMD chip, a galvanometer optical scanner or a polygon scanning mirror, may be used. Both of these technologies, which are commercially available, are known in their application to high speed scanning confocal microscopy. Selection of an appropriate scanning mechanism for use in conjunction with the inventive system and method will be within the level of skill in the art.

According to an exemplary embodiment, the process for fabricating a cell-based artificial cornea follows a 3-step strategy. Referring to FIG. 3, in step 32, we established and optimized culture conditions for growing CEpCs (corneal epithelial cells) and CECs (corneal endothelial cells) on a basement membrane embedded with a nanomesh. After determining the optimal culture conditions, we assembled three corneal layers using 3D live printing, following a layer-by-layer scheme on our 3dLP system. In step 34, the stromal cells are encapsulated in Ac-Col hydrogels (7.5 wt % plus 25 wt % PEGDA) (Acryloyl-PEG-collagen) at a cell density in the range of around 5 million/ml to 25 million/ml stromal cells, which is similar to native cornea. The projection time for printing this layer can be between 1 second to 5 seconds. In step 36, nanomeshes fabricated via 3D nano-printing are embedded in the stromal layer simultaneously. Using the optimized conditions from step 32, the CEC and CEpC layers are assembled with the stroma via two parallel schemes: in steps 38 and 40, the CECs are mixed with an Ac-Col prepolymer solution (5 wt %) and printed with the nanomesh onto the stromal layer via photopolymerization for 30 seconds. In steps 42 and 44, a similar approach may be used to print the CEpC layer on the other side of the stroma. The CEC and CEpC layers need not be concurrently or sequentially printed onto the opposite sides of the stromal layer. Alternatively, pre-developed CEC and CEpC layers, which already have confluent cell layers on their respective nanomesh-incorporated basement membranes, can be “glued” to the stroma by applying a thin film of Ac-Col between the layers and curing via UV exposure (step 46). The final printed constructs are rinsed with saline buffer thoroughly to eliminate any residual unpolymerized solution (step not shown) and further maintained in culture media until transplantation. Finally, the 3D-printed corneas are ready for transplantation and functional assessment.

The following examples provide details of steps of used in an embodiment of the invention:

### Example 1: Growing CEpCs, CECs, and Stromal Cells on a Basement Membrane

Cornea epithelial cells (CECs) undergo continuous renewal from limbal stem or progenitor cells (LSCs), and deficiency in LSCs or corneal epithelium, which turns cornea into a non-transparent, keratinized skin-like epithelium, causes corneal surface disease that leads to blindness. How LSCs are maintained and differentiated into corneal epithelium in healthy individuals, and which molecular events are defective in patients have been largely unknown.

Traditionally, the LSC growth and expansion process requires mouse 3T3 feeder cells, which carry the risk of contamination from animal products, thereby rendering it unsuitable for creating clinically-viable 3D bioprinted corneas. To overcome these obstacles, an in vitro feeder-cell-free, chemically-defined cell culture system to grow LSCs from rabbit and human donors, was developed to enable generation and expansion of a homogeneous population of LSCs, and subsequent differentiation into corneal epithelial cells (CEpCs). This culture system is based on the determination that the transcription factors p63 (tumor protein 63) and PAX6(paired box protein PAX6) act together to specify LSCs, and WNT7A controls corneal epithelium differentiation through PAX6. In the limbal stem cells, WNT7A acts upstream of PAX6 and stimulates its expression via frizzled homolog 5 (FZDS), a receptor for WNT proteins. WNT7A is a secreted morphogen involved in developmental and pathogenic WNT signaling. PAX6 is a transcription factor that controls the fate and differentiation of various eye tissues. RNAi-mediated knockdown of WNT7A or PAX6 induced human limbal stem cells to transition from a corneal to a skin epithelial morphology, a critical defect tightly linked to common human corneal diseases. The WNT7A and PAX6 knockdown cells also had lower expression of corneal keratin 3 (KRT3; CK3) and KRT12 and greater expression of skin epithelial KRT1 and KRT10 than wild-type limbal cells.

Notably, transduction of PAX6 in skin epithelial stem cells is sufficient to convert them to LSC-like cells, and upon transplantation onto eyes in a rabbit corneal injury model, these reprogrammed cells are able to replenish CECs and repair damaged corneal surface. Further details of this process are described in a letter published in Nature, “WNT7A and PAX6 define corneal epithelium homeostatis and pathogenesis”, Nature (2014) doi:10.1038/nature13465), published on-line 2 Jul. 2014, which is incorporated herein by reference. Proliferating LSCs were characterized by expression of P63 and K19, with a high percentage staining positive for the mitotic marker Ki67. We established a 3D LSC differentiation system in which stratified CEpC layers were grown in a basement membrane resembling the Bowman's membrane. Small molecule-ROCK inhibitor Y27632 was used to direct differentiation of LSCs to CEpCs, as evidenced by strong expression of CEpC-specific marker K3/K12.

In parallel, we developed a feeder-cell-free, chemically defined cell culture system containing fibroblast growth factor 2 (FGF2) to grow CEC progenitor cells from human donors. These CEC progenitor cells were then expanded into a homogeneous population of CEC progenitors that were subsequently differentiated into CECs. We observed the hexagonal shape morphology present in native anatomy with strong expression of typical CEC marker ZO-1.

Further, we tested the potential that LSCs cultured on gelatin methacrylate (GelMA) based matrix might be used to treat and repair corneal epithelial defects on a rabbit LSC deficiency model, which mimics a common corneal disease condition in humans. In this test, rabbit GFP-labeled LSCs transplants formed a continuous sheet of epithelial cells with positive staining of corneal specific K3/12 and successfully repaired epithelium defect of the entire corneal surface, and restored and maintained cornea clarity and transparency for over 5 months.

FIGS. 4A-4C illustrate the results of these test: FIG. 4A shows a rabbit cornea post cell transplantation with GFP-labeled LSCs cultured on GelMA based matrix showing typical corneal epithelium histology (left panel: H&E stain) and smooth and transparent cornea surface without epithelial defects (right panel: white light micrograph.) FIG. 4B shows a denuded cornea covered with a human amniotic membrane only. The left panel shows histology of epithelial metaplasia, the right panel shows an opaque cornea with vascularization. FIG. 4C shows a smooth, transparent rabbit cornea three months post transplantation. Cultured GFP+LSCs grown on a GelMA based matrix were used in transplantation experiments, where they were co-stained with K3/12 to show their integration with recipient corneal epithelium.

Corneal stromal cells were also cultured and expanded in vitro. These stromal cells shared similar markers of fibroblast, such as Fibronectin, FSP1 and Vimentin.

### Example 2: 3D Bioprinting

The 3D bioprinting platform offers a rapid biofabrication approach for constructing cell-laden hydrogel scaffolds that 1) have complex user-defined 3D geometries composed of a naturally derived biomaterial; 2) allow for consistent 3D distribution of cells encapsulated within the hydrogel; 3) support cell viability and proliferation; and 4) feature dynamic, multi-scale mechanical cell-scaffold interactions. Importantly, these constructs enable control and integration of complex 3D geometries while providing a physiologically-relevant internal 3D distribution of encapsulated cells. Through such precise control of spatial and temporal distributions of biological factors in 3D scaffolds, we are able to evaluate the interactions of cells with extracellular matrix (ECM) proteins at the nanometer length scale, with the ultimate goal of creating advanced, clinically translatable biomimetic scaffolds.

Using 3D bioprinting, artificial corneas are fabricated using the same dimension and curvature of the native cornea to replicate the patient's cornea. The naturally derived material can support cell growth within the construct and recruit host cells for better integration of the constructs. Due to the high efficiency of the 3D printing technology, a few seconds is sufficient for one layer. Therefore, it is possible to maintain a highly homogenous cell distribution within each layer. In addition, spatial localization of different cell types can be precisely controlled, which is critical for corneal function. For example, we can fabricate small features around 5 microns, i.e., smaller than a cell. With this resolution, we can control the spatial localization of very small cell population, even single cell. By using materials of different degradation profile, we can guide the cell migration and thus control their temporal distribution. By patterning growth factors within the constructs, we can also modulate the cell proliferation/differentiation, and manage the cell distribution.

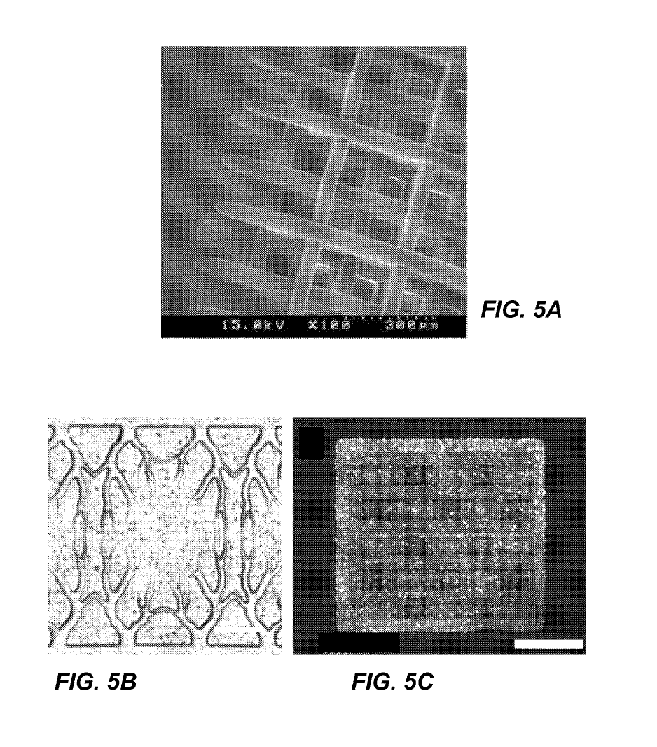

FIGS. 5A-C show exemplary microstructures created by 3D bioprinting: FIG. 5A, a multi-layer log-pile scaffold with 200 μm pore size using PEGDA; FIG. 5B, a 3D-printed vasculature-like microstructure in GelMA (scale bar=30 μm); FIG. 5C, 10 T1/2 cells encapsulated in a GelMA scaffold remain viable and proliferative at 8 hours after encapsulation, assessed via a calcein-AM/ethidium homodimer LIVE/DEAD assay (scale bar=1 mm).

### Example 3: Biomaterials for Cornea Tissues

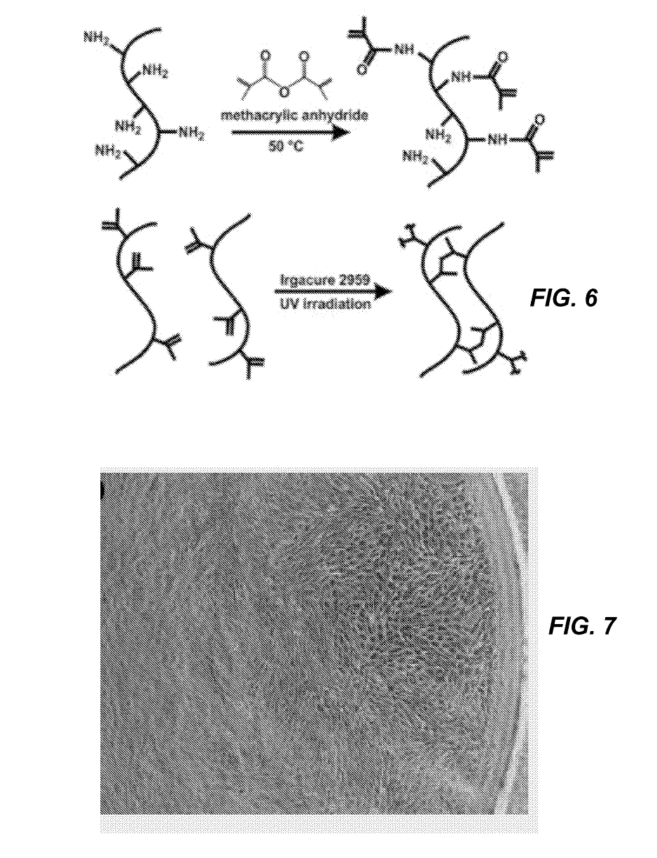

Collagen has been used extensively as a biomaterial for corneal tissue engineering, as it comprises the main component of corneal extracellular matrix (ECM). Collagen, as a matrix constituent, has been demonstrated to support epithelial cells in forming a protective layer and to promote re-innervation by neurons. A chemically-crosslinked biosynthetic collagen matrix has shown significant promise in a phase I clinical trial. In order to modulate the degradation and mechanical properties of a collagen matrix, most studies have used chemical crosslinking approaches, which are largely incompatible with cell encapsulation. Acryloyl-PEG-collagen (Ac-Col) offers an excellent alternative for corneal tissue engineering due to its biocompatibility, optical properties, and ability for photopolymerization. Preliminary tests have been performed to assess the optical properties of a stromal cell-laden film made of GelMA, which is an Ac-Col analogue. FIG. 6 illustrates an exemplary synthesis scheme for GelMA hydrogels. CECs were seeded and cultivated on an optically transparent corneal stroma fabricated with GelMA using the 3dLP system. Even after the formation of a confluent CEC cell sheet, shown in FIG. 7, the transparency of the construct was maintained.

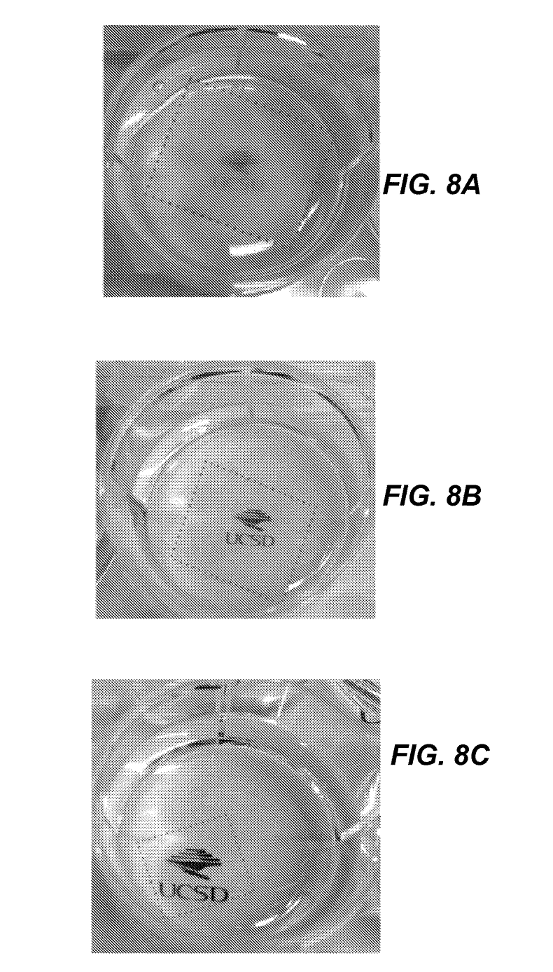

Evaluation of the impact on optical transparency of varied hybrid hydrogel combinations and exposure times was performed. FIGS. 8A-8C illustrate the results, in which the optical clarity of the UCSD logo viewed through the fabricated structure is compared for each combination. FIG. 8A exhibits decreased transparency for 7.5 wt % GelMA (gelatin methacrylate) with 1 wt % MA-HA (methacrylate-hyaluronic acid) (MW=200 KDa), UV exposure=1 minute. Improvement in transparency was achieved with 7.5 wt % GelMA, 1 wt % MA-HA (MW=200 KDa) and 2.5% PEGDA (poly (ethylene glycol) diacrylate) (MW=700 KDa), UV exposure=30 seconds, as shown in FIG. 8B. Still better transparency was obtained using 7.5 wt % GelMA, 2.5 wt % MA-HA (MW=200 KDa) and 2.5% PEGDA (MW=700 KDa) with UV exposure=30 seconds. These results indicate that clarity increases as the MA-HA concentration increases from 1 wt % to 2.5 wt %.

Several material compositions have been tested and the optical property of most of the material choices is very good. In one example, with 7.5 wt % GelMA or Ac-Col and 25 wt % PEGDA plus 0.075 wt % LAP (lithium phenyl-2,3,6-trimethylbenzoylphosphinate) as photoinitiator, produced a transparent film that exhibited comparable absorbance to that of PBS solution in the range of 280 nm to 1000 nm. The UV exposure time does not appear to affect the transparency of this film. In terms of MA-HA, 7.5 wt % GelMA with 2.5 wt % MA-HA and 2.5% PEGDA provides excellent optical properties as well after 30 seconds of UV exposure.

As is known in the art, because most photoinitiators are cytotoxic. Selection of the type and concentration of photoinitiator to obtain the desired film properties while maintaining cell viability will be within the level of skill in the art.

### Example 4: Transplantation of 3D-printed Corneas

Three corneal layers were fabricated using 3D live printing as described above. Specifically, a PEGDA nanomesh was embedded in acryloyl-PEG-collagen to support the corneal stroma. The CEpC layer and CEC layer were built on each side of the stroma layer. The resulting bioprinted cornea was transplanted onto a rabbit recipient eye.

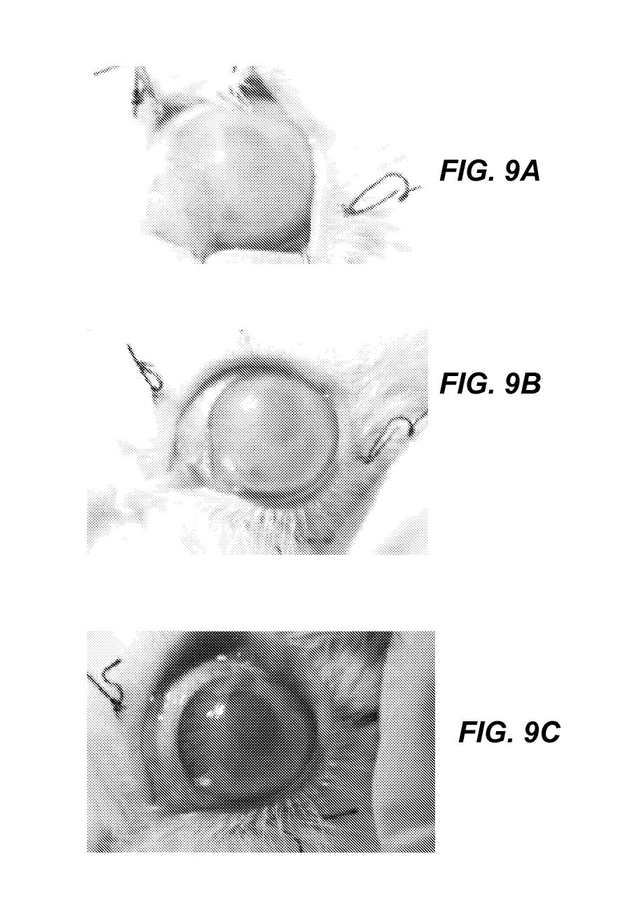

New Zealand white rabbits were anaesthetized with intramuscular injection of xylazine hydrochloride (2.5 mg/ml) and ketamine hydrochloride (37.5 mg/ml). A corneal recipient stromal bed with a reverse-button like structure was created in the recipient eye using a femtosecond laser machine (Zeiss). The bioprinted corneal donor tissue was cut into a button-shape structure to fit onto the prepared recipient stromal bed. The surface was then covered by a human amniotic membrane (Bio-tissue), which was secured with 10.0 VICRYL sutures (Ethicon) to the recipient conjunctiva. FIGS. 9A and 9B show the gradual recovery of clarity and functionality post-transplant at day 5 and day 10, respectively. A gradual decrease in corneal edema and increase in cornea clarity was observed at day 15 post transplantation, shown in FIG. 9C, indicating functional recovery of corneal endothelium. The corneal surface epithelium was observed to be smooth and intact, indicating functional transplanted CEpCs.

According to the embodiments described herein, the use of 3D bioprinting technology allows for cell encapsulation, enabling live printing of tissue structures with micro and nanometer resolution. The cell-laden corneal substitutes can reduce the amount of time required for the transplants to integrate with the host tissue. In addition, the digital (i.e., customizable) nature of 3D printing allows development of patient-specific tissue models with designed shape and curvature. The custom shape and curvature can be designed according to the patient's native cornea.

Using procedures that are known in the art, corneal topography measurements can be obtained for the patient prior the transplant procedure. For example, instruments used in clinical practice most often are based on Placido reflective image analysis, which uses the analysis of reflected images of multiple concentric rings projected on the cornea to obtain keratometric dioptric range and surface curvature. Using the clinical data generated by such testing, computer software can be used to generate patient specific corneal design, which will then be fabricated using the 3D printing platform. A layer by layer printing approach may be used. In some cases, in order to generate highly complex corneal geometries, it may be appropriate to utilize a non-linear 3D printing scheme such as that disclosed in PCT Application No. PCT/US2015/050522, filed Sep. 16, 2015, which is incorporated herein by reference.

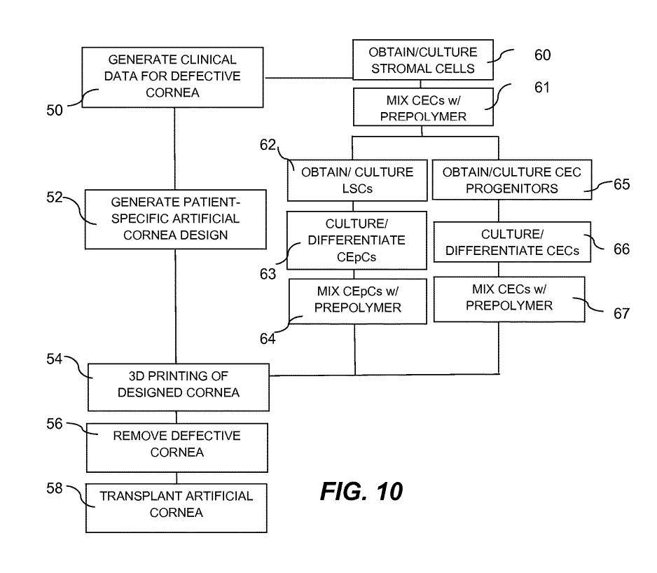

FIG. 10 summarizes an exemplary procedure for design, fabrication and transplantation of an artificial cornea according to an embodiment of the invention. Starting with a determination that replacement of the cornea is medically necessary, in step 50, data is generated using clinical instrumentation for measurement of the patient's cornea. Using computer-aided design software, in step 52, a sequence of printing steps is developed to control the 3dLP printer to fabricate an artificial cornea to the correct dimensions and desired characteristics for the patient's eye. In parallel to creation of the computer control program for printing the patient-specific cornea, stromal cells and LSCs are cultured and mixed into a prepolymer solution in steps 60 through 67. While not being limited to use of a patient's own cells, the use of autologous tissue as the source of stromal cells, progenitor CECs, and/or LSCs can provide a further advantage of reducing or eliminating the possible need for immunosuppression. In steps 63 and 66 respectively, the LSCs are differentiated into CEpCs and CEC progenitors from human donors are differentiated into CECs. In steps 61, 64 and 67 the cultured cells are each mixed into prepolymer solutions. (It should be noted that while the flow diagram shows the stromal layers being prepared before formation of the CEC and CEpC layers, one or more of the three layers can be printed at different times, e.g., in advance, or they can be printed in parallel i.e., not in a particular sequence, and assembled as described above.) In step 54, the cultured stromal cells, CECs and CEpCs are incorporated into their respective layers as describe above. They may be printed sequentially or printed separately and assembled from separately printed layers to define the CEC-stromal-CEpC layered structure of the cornea. The defective cornea is removed in step 56 using procedures known in the art, and the stromal bed is prepared to receive the transplant, followed by transplantation of the artificial cornea in step 58.

3D-printed cornea tissues fabricated according to the procedures described herein will have immediate applications in clinical transplantation, human ocular surface disease modeling (e.g., for dry eye diseases), early drug screening to replace or reduce the need for animal testing, and in drug efficacy testing for wound healing. This technology provides a strong basis for the development of temporary or permanent cornea replacements. The embodiments described herein could lead to readily available, complex engineered tissues that recapitulate the functionality of their natural human counterparts and are suitable for clinical adoption as well as emerging biomedical research.

### REFERENCES (INCORPORATED HEREIN BY REFERENCE)

1. Fagerholm P, Lagali NS, Merrett K, Jackson WB, Munger R, Liu Y, Polarek JW, Soderqvist M, and Griffith M. A biosynthetic alternative to human donor tissue for inducing corneal regeneration: 24-month follow-up of a phase 1 clinical study. Sci Transl Med. 2010;2(46):46ra61

2. Myung D, Duhamel PE, Cochran JR, Noolandi J, Ta CN, and Frank CW. Development of hydrogel-based keratoprostheses: a materials perspective. Biotechnol Prog. 2008;24(3):735-41

3. Crabb RA, Chau EP, Evans MC, Barocas VH, and Hubel A. Biomechanical and microstructural characteristics of a collagen film-based corneal stroma equivalent. Tissue Eng. 2006;12(6):1565-75.

1. A method for fabricating an artificial cornea, comprising:
culturing live stromal cells;
3D bioprinting a stromal layer encapsulating the live stromal cells into a first hydrogel nanomesh;
culturing live corneal endothelial cells (CECs);
3D bioprinting a CEC layer encapsulating the live CECs into a second hydrogel nanomesh;
culturing live corneal epithelial cells (CEpCs);
3D bioprinting a CEpC layer encapsulating the live CEpCs into a third hydrogel nanomesh; and
attaching the CEC layer to a first side of the stromal layer and the CEpC layer to a second side of the stromal layer.

2. The method of claim 1, wherein the steps of culturing are performed in parallel.

3. The method of claim 1, wherein the steps of 3D bioprinting the CEC layer and the CEpC layers are performed in parallel.

4. The method of claim 1, wherein the step of attaching the CEC layer to the first side of the stromal layer comprises sequentially printing the stromal layer and the CEC layer.

5. The method of claim 1, wherein the step of attaching the CEC layer to the first side of the stromal layer comprises applying a thin film of hydrogel between each of the layers and curing via UV exposure.

6. The method of claim 1, wherein the step of attaching the CEpC layer to the second side of the stromal layer comprises applying a thin film of hydrogel between each of the layers and curing via UV exposure.

7. The method of claim 1, further comprising, prior to 3D bioprinting the CEC layer, mixing the CECs with a prepolymer solution of acryloyl-PEG-collagen.

8. The method of claim 7, wherein the prepolymer solution further comprises MA-HA.

9. The method of claim 1, further comprising, prior to 3D bioprinting the CEpC layer, mixing the CEpCs with a prepolymer solution of acryloyl-PEG-collagen.

10. The method of claim 9, wherein the prepolymer solution further comprises MA-HA.

11. The method of claim 1, further comprising, prior to 3D bioprinting the stromal layer, encapsulating the stromal cells in an acryloyl-PEG-collagen hydrogel.

12. The method of claim 11, wherein the prepolymer solution further comprises MA-HA.

13. The method of claim 11, wherein the stromal cells are encapsulated at a cell density in the range of around 5 million/ml to 25 million/ml stromal cells.

14. The method of claim 1, wherein the step of culturing live CEpCs comprises culturing LSCs, and differentiating the LSCs into CEpCs.

15. The method of claim 14, wherein the LSCs are obtained from autologous tissue.

16. The method of claim 1, wherein the step of culturing live CECs comprises culturing CEC progenitors from a human donor, and differentiating the CEC progenitors into CECs.

17. The method of claim 14, wherein the CEC progenitors are obtained from autologous tissue.

18. The method of claim 1, wherein the first, second and third hydrogel nanomeshes each comprise PEGDA.

19. An artificial cornea, comprising:
a layered structure comprising:
a 3D bioprinted stromal layer comprising live stromal cells encapsulated into a first hydrogel nanomesh, the stromal layer having a first side and a second side;
a 3D bioprinted CEC layer comprising live corneal endothelial cells (CECs) encapsulated into a second hydrogel nanomesh;
a 3D bioprinted CEpC layer comprising live corneal epithelial cells (CEpCs) encapsulated into a third hydrogel nanomesh; and
wherein the CEC layer is attached to the first side of the stromal layer and the CEpC layer is attached to the second side of the stromal layer.

20. The artificial cornea of claim 19, wherein one or more of the CEC layer and the CEpC layer is attached by a thin film of hydrogel applied between the layers and cured via UV exposure.

21. The artificial cornea of claim 19, wherein live cells are encapsulated into a hydrogel material prior to bioprinting the corresponding one of the stromal layer, the CEC layer, and the CEpC layer.

22. The artificial cornea of claim 21, wherein the hydrogel comprises acryloyl-PEG-collagen.

23. The artificial cornea of claim 20, wherein the hydrogel further comprises MA-HA.

24-26. (canceled)

27. The artificial cornea of claim 19, wherein the live CEpCs comprise cultured and differentiated LSCs.

28-30. (canceled)
